(19) United States
(12) Patent Application Publication
(10) Pub. No.: US 2018/0055579 A1
(43) Pub. Date: Mar. 1, 2018

(54) CALLIBRATION-FREE SYSTEM AND METHOD FOR DETERMINING THE THREE-DIMENSIONAL LOCATION AND ORIENTATION OF IDENTIFICATION MARKERS

(71) Applicant: Navigate Surgical Technologies, Inc., Vancouver (CA)

(72) Inventors: Ehud (Udi) DAON, North Vancouver (CA); Martin Gregory BECKETT, Bowen Island (CA)

(21) Appl. No.: 15/788,826

(22) Filed: Oct. 20, 2017

Related U.S. Application Data
(63) Continuation-in-part of application No. 13/822,358, filed on Mar. 12, 2013.
(60) Provisional application No. 62/466,996, filed on Mar. 3, 2017.
(60) Provisional application No. 62/142,194, filed on Apr. 2, 2015.
(60) Provisional application No. 61/533,058, filed on Sep. 9, 2011.
(60) Provisional application No. 61/616,718, filed on Mar. 28, 2012.
(60) Provisional application No. 61/616,673, filed on Mar. 28, 2012.

(51) Int. Cl. A61B 34/20; A61B 90/00; A61C 1/00

(52) U.S. Cl. CPC A61B 34/20; A61B 90/39; A61B 2090/3983; A61B 2034/2055; A61B 2090/3966; A61C 1/0007

(57) ABSTRACT

The present invention involves a vectorized multi-material fiducial reference for use in tracking on a user-calibration free basis a non-visible scan-detectable structure of a body as part of a three-dimensional tracking system. The fiducial consists of scan-detectable elements embedded within the body of the fiducial, the body being formed of a material compatible with the tracked body. The scan-detectable elements are embedded in a rotationally asymmetric pattern in the fiducial with to an accuracy compatible with surgery. A vectorized tracking marker may be attached directly or indirectly to the fiducial. The system employs a controller in communication with a non-stereo optical tracker to track in real time the marker and thereby the fiducial based on a prior scan of the surgical site with the fiducial attached. A method for manufacturing the fiducial employs pins to hold the scan-detectable elements accurately in position while forming the body of the fiducial around the elements.

### CROSS REFERENCE TO RELATED APPLICATIONS

The present application claims the benefit under 35 U.S.C. § 119(e) of provisional applications 62/466,996 and 62/142,194, filed Mar. 3, 2017 and Oct. 24, 2016, respectively, and claims the benefit under 35 U.S.C § 120 as a continuation-in-part of U.S. patent application Ser. No. 13/822,358, filed Mar. 13, 2013, which is the United States National Stage application under 35 U.S.C. § 371 of International Patent Application PCT/IL2012/00036, filed Oct. 23, 2012, which claims the benefit under 35 U.S.C. § 119(e) of Provisional Patent Applications Ser. Nos. 61/533,058; 61/616,718; and 61/616,673; filed on Oct. 28, 2011; Mar. 28, 2012; and Mar. 28, 2012, respectively, the disclosures of which are incorporated by reference in their entirety.

### BACKGROUND OF THE INVENTION

### Field of the Invention

The invention relates to location monitoring hardware and software systems. More specifically, the field of the invention is that of surgical equipment and software for monitoring surgical conditions.

### Description of the Related Art

Visual and other sensory systems are known, with such systems being capable of both observing and monitoring surgical procedures. With such observation and monitoring systems, computer aided surgeries are now possible, and in fact are being routinely performed. In such procedures, the computer software interacts with both clinical images of the patient and observed surgical images from the current surgical procedure to provide guidance to the physician in conducting the surgery. For example, in one known system a carrier assembly bears at least one fiducial marker onto an attachment element in a precisely repeatable position with respect to a patient's jaw bone, employing the carrier assembly for providing registration between the fiducial marker and the patient's jaw bone and implanting the tooth implant by employing a tracking system which uses the registration to guide a drilling assembly. With this relatively new computer implemented technology, further improvements may further advance the effectiveness of surgical procedures.

### SUMMARY OF THE INVENTION

The present invention involves embodiments of surgical hardware and software monitoring system and method which allows for surgical planning while the patient is available for surgery, for example while the patient is being prepared for surgery so that the system may model the surgical site. In one embodiment, the model may be used to track contemplated surgical procedures and warn the physician regarding possible boundary violations that would indicate an inappropriate location in a surgical procedure. In another embodiment, the hardware may track the movement of instruments during the procedure and in reference to the model to enhance observation of the procedure. In this way, physicians are provided an additional tool to improve surgical planning and performance.

The system uses a particularly configured fiducial reference, to orient the monitoring system with regard to the critical area. The fiducial reference is attached to a location near the intended surgical area. For example, in the example of a dental surgery, a splint may be used to securely locate the fiducial reference near the surgical area. The fiducial reference may then be used as a point of reference, or a fiducial, for the further image processing of the surgical site. The fiducial reference may be identified relative to other portions of the surgical area by having a recognizable fiducial marker apparent in the scan.

The embodiments of the invention involve automatically computing the three-dimensional location of the patient by means of a tracking device that may be a tracking marker. The tracking marker may be attached in fixed spatial relation either directly to the fiducial reference, or attached to the fiducial reference via a tracking pole that itself may have a distinct three-dimensional shape. In the dental surgery example, a tracking pole is mechanically connected to the base of the fiducial reference that is in turn fixed in the patient's mouth. Each tracking pole device has a particular observation pattern, located either on itself or on a suitable tracking marker, and a particular geometrical connection to the base, which the computer software recognizes as corresponding to a particular geometry for subsequent location calculations. Although individual tracking pole devices have distinct configurations, they may all share the same connection base and thus may be used with any fiducial reference. The particular tracking information calculations are dictated by the particular tracking pole used, and actual patient location is calculated accordingly. Thus, tracking pole devices may be interchanged and calculation of the location remains the same. This provides, in the case of dental surgery, automatic recognition of the patient head location in space. Alternatively, a sensor device, or a tracker, may be in a known position relative to the fiducial key and its tracking pole, so that the current data image may be mapped to the scan image items.

The fiducial reference and each tracking pole or associated tracking marker may bear a pattern, made of radio opaque material in the case of the fiducial. When imaging information or previous scans of the surgical site are interpreted by the software, the particular items are recognized. Typically, each instrument used in the procedure has a unique pattern on its associated tracking marker so that the tracker information identifies the instrument. The software creates a model of the surgical site, in one embodiment a coordinate system, according to the location and orientation of the patterns on the fiducial reference and/or tracking pole(s) or their attached tracking markers. By way of example, in the embodiment where the fiducial reference has an associated pre-assigned pattern, analysis software interpreting image information from the tracker may recognize the pattern and may select the site of the base of the fiducial to be at the location where the fiducial reference is attached to a splint. If the fiducial key does not have an associated pattern, a fiducial site is designated. In the dental example this may be at a particular spatial relation to the tooth, and a splint location may be automatically designed for placement of the fiducial reference.

In a first aspect of the invention there is provided a surgical monitoring system comprising a vectorized fiducial reference configured for removably attaching to a location proximate a surgical site, for having a three-dimensional location and orientation determinable based on scan data of the surgical site, and for having the three-dimensional location and orientation determinable based on image information about the surgical site; a tracker arranged for obtaining the image information; and a controller configured for spatially relating the image information to the scan data and for determining the three-dimensional location and orientation of the fiducial reference. In one embodiment of the invention the fiducial reference may be rigidly and removably attachable to a part of the surgical site. In such an embodiment the fiducial reference may be repeatably attachable in the same three-dimensional orientation to the same location on the particular part of the surgical site.

The vectorized fiducial reference is at least one of marked and shaped for having at least one of its location and its orientation determined from the scan data and to allow it to be uniquely identified from the scan data. The surgical monitoring system further comprises a first vectorized tracking marker in fixed three-dimensional spatial relationship with the fiducial reference, wherein the first tracking marker is configured for having at least one of its location and its orientation determined by the controller based on the image information and the scan data. The first tracking marker may be configured to be removably and rigidly connected to the fiducial reference by a first tracking pole. The first tracking pole may have a three-dimensional structure uniquely identifiable by the controller from the image information. The three-dimensional structure of the first tracking pole allows its three-dimensional orientation of the first tracking pole to be determined by the controller from the image information.

The first tracking pole and fiducial reference may be configured to allow the first tracking pole to connect to a single unique location on the fiducial reference in a first single unique three-dimensional orientation. The fiducial reference may be configured for the attachment in a single second unique three-dimensional orientation of at least a second tracking pole attached to a second tracking marker. The first tracking marker may have a three-dimensional shape that is uniquely identifiable by the controller from the image information. The first tracking marker may have a three-dimensional shape that allows its three-dimensional orientation to be determined by the controller from the image information. The first tracking marker may have a marking that is uniquely identifiable by the controller and the marking may be configured for allowing at least one of its location and its orientation to be determined by the controller based on the image information and the scan data.

The surgical monitoring system may comprise further vectorized tracking markers attached to implements proximate the surgery site and the controller may be configured for determining locations and orientations of the implements based on the image information and information about the further tracking markers.

In another aspect of the invention there is provided a method for relating in real time the three-dimensional location and orientation of a surgical site on a patient to the location and orientation of the surgical site in a scan of the surgical site, the method comprising removably attaching a vectorized fiducial reference to a fiducial location on the patient proximate the surgical site; performing the scan with the fiducial reference attached to the fiducial location to obtain scan data; determining the three-dimensional location and orientation of the fiducial reference from the scan data; obtaining real time image information of the surgical site; determining in real time the three-dimensional location and orientation of the fiducial reference from the image information; deriving a spatial transformation matrix or expressing in real time the three-dimensional location and orientation of the fiducial reference as determined from the image information in terms of the three-dimensional location and orientation of the fiducial reference as determined from the scan data.

The obtaining of real time image information of the surgical site may comprise rigidly and removably attaching to the fiducial reference a first vectorized tracking marker in a fixed three-dimensional spatial relationship with the fiducial reference. The first tracking marker may be configured for having its location and its orientation determined based on the image information. The attaching of the first tracking marker to the fiducial reference may comprise rigidly and removably attaching the first tracking marker to the fiducial reference by means of a tracking pole. The obtaining of the real time image information of the surgical site may comprise rigidly and removably attaching to the fiducial reference a tracking pole in a fixed three-dimensional spatial relationship with the fiducial reference, and the tracking pole may be vectorized in having a distinctly identifiable three-dimensional shape that allows its location and orientation to be uniquely determined from the image information.

In yet a further aspect of the invention there is provided a method for real time monitoring the position of an object in relation to a surgical site of a patient, the method comprising removably attaching a vectorized fiducial reference to a fiducial location on the patient proximate the surgical site; performing a scan with the fiducial reference attached to the fiducial location to obtain scan data; determining the three-dimensional location and orientation of the fiducial reference from the scan data; obtaining real time image information of the surgical site; determining in real time the three-dimensional location and orientation of the fiducial reference from the image information; deriving a spatial transformation matrix for expressing in real time the three-dimensional location and orientation of the fiducial reference as determined from the image information in terms of the three-dimensional location and orientation of the fiducial reference as determined from the scan data; determining in real time the three-dimensional location and orientation of the object from the image information; and relating the three-dimensional location and orientation of the object to the three-dimensional location and orientation of the fiducial reference as determined from the image information. The determining in real time of the three-dimensional location and orientation of the object from the image information may comprise rigidly attaching a vectorized tracking marker to the object.

In one alternative embodiment, the tracker itself is attached to the fiducial reference so that the location of an object having a vectorized marker may be observed from a known position.

In a further aspect, the system may be configured as a robotic surgery system. The controller may control a robotic surgery instrument, guiding it to execute the surgical process based on image information from the tracker. The image information of a tracking marker allows determination of the three-dimensional pose of the fiducial marker for which a prior scan has provided scan data for use by the controller. Computer software stored in a memory of the controller is executed in a processor of the controller to guide the instrument. The instrument may be a biopsy needle. The controller may operate on an autonomous basis, with human intervention being optional. The fiducial remains rigidly attached to the surgical site, and the marker remains in its fixed relative position and orientation with respect to fiducial if and when the patient moves. With both markers tracked by the tracker, the controller may autonomously guide the robotic instrument despite the motion of the patient. In cases where the fiducial reference is directly visible to the tracker the fiducial may itself be vectorized with suitable markers bearing patterns that allow the spatial position and orientation of the fiducial to be directly tracked by the tracker without requiring separate tracking markers to be attached to the fiducial tracking poles.

In a further aspect, a method is provided for guiding at a surgical site a robotic surgery instrument, the method comprising providing proximate the surgical site the robotic surgery instrument bearing in fixed three-dimensional spatial relationship with the instrument a first passive vectorized tracking marker, the marker bearing at least one first identifiably unique rotationally asymmetric pattern; disposing a non-stereo optical tracker to obtain image information of the surgical site and the instrument; obtaining image information about the surgical site from the non-stereo optical tracker; obtaining geometric information from a database, the geometric information comprising information about the first tracking marker; identifying the first tracking marker in the image information on the basis of the at least one first unique pattern; determining within the image information the location of at least one first pattern reference point of the first tracking marker based on the geometric information; determining within the image information the rotational orientation of the first tracking marker based on the geometric information; and guiding the robotic surgery instrument based on the location of the at least one first pattern reference point and the rotational orientation of the first tracking marker.

In some implementations of the method, the fiducial reference may directly bear the second tracking marker, so that the step of attaching to the fiducial reference the second tracking marker in fixed three-dimensional spatial relationship with the fiducial reference is obviated.

In a further aspect, a user-calibration-free tracking system is provided for monitoring the position and orientation of non-visible scan-detectable structure of a body of interest, the system comprising: a vectorized fiducial reference adapted to be rigidly attached to the body of interest, the fiducial reference comprising a structural body composed of a structural material compatible with a material of the body of interest and one or more scan-detectable elements composed of a scan-detectable material rigidly embedded in the structural material wherein the one or more scan-detectable elements comprise a rotationally non-symmetric pattern; a passive vectorized tracking marker rigidly attached to the fiducial reference at a predetermined location in a predetermined three-dimensional orientation with respect to the fiducial reference; a non-stereo optical tracker arranged to obtain image information about an area encompassing at least a portion of the tracking marker; a controller in communication with the tracker; a display system in communication with the controller; and previously obtained scan data of the body of interest with the fiducial reference fixed to the body showing the scan-detectable elements relative to the non-visible structure of the body of interest; wherein the controller comprises a processor, a memory and a software program having a series of instructions which when executed by the processor determine the relative position and orientation of the marker and the one or more scan-detectable elements based on the image information and the scan data. The tracking marker may be removably attached to the fiducial reference and may be attached to the fiducial reference via a tracking pole.

The system may further comprise a database, the database containing: geometric information about the tracking marker; and information about the rotationally non-symmetric pattern of the one or more scan-detectable elements.

In a further aspect a fiducial reference is provided for use in tracking a non-visible scan-detectable structure of a body of interest, the fiducial reference comprising: a structural body composed of a structural material compatible with a material of the body of interest; and one or more scan-detectable elements composed of a scan-detectable material rigidly embedded in the structural material; wherein the one or more scan-detectable elements comprise a rotationally non-symmetric pattern. The one or more scan-detectable elements may be embedded in the structural material with 100% precision to an accuracy compatible with one of human and animal surgery. The accuracy may be a distance of 150 microns or less. In other cases the accuracy may be a distance of 80 microns or less. In yet other cases the accuracy may be a distance of 40 microns or less. More particularly, the accuracy may be a distance of 16 microns or less.

The scan-detectable material may have a radiographic density approximating a radiographic density of one of human and animal bone. The scan-detectable material may be one of a metal, a metallic-oxide ceramic, and silicon nitride. More specifically, the scan-detectable material may be one of stainless steel, titanium, aluminum oxide, and zirconium oxide.

The fiducial reference may further comprise a vectorized tracking marker. The tracking marker may further bear an optically detectable rotationally asymmetric pattern. The fiducial reference may further comprise a locating hole for rigidly and removably attaching a vectorized tracking marker. The tracking marker may bear an optically detectable rotationally asymmetric pattern. The tracking marker may be attachable to the fiducial by means of a tracking pole.

In a further aspect, a method is provided for manufacturing a multi-material fiducial reference for tracking a non-visible scan-detectable structure of a body of interest, the method comprising: providing one or more scan-detectable elements; providing a mold shaped to receive the one or more scan-detectable elements and an injection moldable material compatible with the body of interest; rigidly positioning in a predetermined position and orientation within the mold the one or more scan-detectable elements by means of pins to an accuracy of at least 150 microns; and injecting the injection moldable material into the mold while rigidly holding the scan-detectable elements by means of the pins. The method may further comprise removing the pins; and further injecting additional injection moldable material to surround the scan-detectable elements.

### BRIEF DESCRIPTION OF THE DRAWINGS

The abovementioned and other features and objects of this invention, and the manner of attaining them, will become more apparent and the invention itself will be better understood by reference to the following description of an embodiment of the invention taken in conjunction with the accompanying drawings, wherein:

FIG. 1 is a schematic diagrammatic view of a network system in which embodiments of the present invention may be utilized.

FIG. 2 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized as controller and display in conjunction with embodiments of the present invention.

FIGS. 3A-M are drawings of hardware components of the surgical monitoring system and patterns of markings on the components according to embodiments of the invention.

FIGS. 4A-C is a flow chart diagram illustrating one embodiment of the registering method of the present invention.

FIG. 5 is a drawing of a dental passive vectorized fiducial key with a tracking pole and a dental drill according to one embodiment of the present invention.

FIG. 6 is a drawing of an endoscopic surgical site showing the passive vectorized fiducial key, endoscope, and biopsy needle according to another embodiment of the invention.

FIG. 7 is a drawing of a flow chart for a method of establishing a coordinate system at a passive vectorized fiducial key according to an embodiment of the present invention.

FIGS. 8a and 8b together present a flow chart of a method for guiding at a surgical site a robotic surgery instrument.

FIG. 9 is a flow chart of a method for manufacturing a multi-material fiducial.

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiments disclosed below are not intended to be exhaustive or limit the invention to the precise form disclosed in the following detailed description. Rather, the embodiments are chosen and described so that others skilled in the art may utilize their teachings.

The detailed descriptions that follow are presented in part in terms of algorithms and symbolic representations of operations on data bits within a computer memory representing alphanumeric characters or other information. The hardware components are shown with particular shapes and relative orientations and sizes using particular scanning techniques, although in the general case one of ordinary skill recognizes that a variety of particular shapes and orientations and scanning methodologies may be used within the teaching of the present invention. A computer generally includes a processor for executing instructions and memory for storing instructions and data, including interfaces to obtain and process imaging data. When a general-purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities, observing and measuring scanned data representative of matter around the surgical site. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed to capture the underlying data of an image. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements that impart or manifest a physical organization on the information stored in memory. More than mere abstraction, the data structures are specific electrical or magnetic structural elements in memory, which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein that form part of the present invention; the operations are machine operations. Useful machines for performing the operations of the present invention include general-purpose digital computers or other similar devices. In all cases the distinction between the method operations in operating a computer and the method of computation itself should be recognized. The present invention relates to a method and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

The present invention also relates to an apparatus for performing these operations. This apparatus may be specifically constructed for the required purposes or it may comprise a general-purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipments through signals configured to particular protocols, which may or may not require specific hardware or programming to interact. In particular, various general-purpose machines may be used with programs written in accordance with the teachings herein, or it may prove more convenient to construct more specialized apparatus to perform the required method steps. The required structure for a variety of these machines will appear from the description below.

The present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects. Often, but not necessarily, a physical object has a corresponding software object that may collect and transmit observed data from the physical device to the software system. Such observed data may be accessed from the physical object and/or the software object merely as an item of convenience; therefore where “actual data” is used in the following description, such “actual data” may be from the instrument itself or from the corresponding software object or module.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and here the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system may be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions, which may include sending additional messages to one or more other objects. The other objects may in turn carry out additional functions in response to the messages they receive. Including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms that are used frequently have specialized meanings in the present context. The term “object” relates to a set of computer instructions and associated data, which may be activated directly or indirectly by the user. The terms “windowing environment”, “running in windows”, and “object oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers that are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks may access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a worldwide network of computers, namely the “World Wide Web” or simply the “Web”. Examples of Browsers compatible with the present invention include the Internet Explorer program sold by Microsoft Corporation (Internet Explorer is a trademark of Microsoft Corporation), the Opera Browser program created by Opera Software ASA, or the Firefox browser program distributed by the Mozilla Foundation (Firefox is a registered trademark of the Mozilla Foundation). Although the following description details such operations in terms of a graphic user interface of a Browser, the present invention may be practiced with text based interfaces, or even with voice or visually activated interfaces, that have many of the functions of a graphic based Browser.

Browsers display information, which is formatted in a Standard Generalized Markup Language (“SGML”) or a HyperText Markup Language (“HTML”), both being scripting languages, which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an eXtensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method).

The terms “personal digital assistant” or “PDA”, as defined above, means any handheld, mobile device that combines computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

In wireless wide area networks, communication primarily occurs through the transmission of radio signals over analog, digital cellular, or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (CDPD”) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system, which allows for application programs to be implemented on a mobile device such as a mobile telephone or PDA. Examples of Mobile Software are Java and Java ME (Java and JavaME are trademarks of Sun Microsystems, Inc. of Santa Clara, Calif.), BREW (BREW is a registered trademark of Qualcomm Incorporated of San Diego, Calif.), Windows Mobile (Windows is a registered trademark of Microsoft Corporation of Redmond, Wash.), Palm OS (Palm is a registered trademark of Palm, Inc. of Sunnyvale, Calif.), Symbian OS (Symbian is a registered trademark of Symbian Software Limited Corporation of London, United Kingdom), ANDROID OS (ANDROID is a registered trademark of Google, Inc. of Mountain View, Calif.), and iPhone OS (iPhone is a registered trademark of Apple, Inc. of Cupertino, Calif.), and Windows Phone 7. “Mobile Apps” refers to software programs written for execution with Mobile Software.

The terms “scan, fiducial reference”, “fiducial location”, “marker,” “tracker” and “image information” have particular meanings in the present disclosure. For purposes of the present disclosure, “scan” or derivatives thereof refer to x-ray, magnetic resonance imaging (MRI), computerized tomography (CT), sonography, cone beam computerized tomography (CBCT), or any system that produces a quantitative spatial representation of a patient and a “scanner” is the means by which such scans are obtained. The term “fiducial reference”, “fiducial key”, or simply “fiducial” refers to an object or reference on the image of a scan that is uniquely identifiable as a fixed recognizable point. In the present specification the term “fiducial location” refers to a useful location to which a fiducial reference is attached. A “fiducial location” will typically be proximate a surgical site. The term “marker” or “tracking marker” refers to an object or reference that may be perceived by a sensor proximate to the location of the surgical or dental procedure, where the sensor may be an optical sensor, a radio frequency identifier (RFID), a sonic motion detector, an ultra-violet or infrared sensor. The term “tracker” refers to a device or system of devices able to determine the location of the markers and their orientation and movement continually in ‘real time’ during a procedure. As an example of a possible implementation, if the markers are composed of printed targets then the tracker may include a stereo camera pair. In some embodiments, the tracker may be a non-stereo optical tracker, for example a camera. The camera may, for example, operate in the visible or near-infrared range. The term “image information” is used in the present specification to describe information obtained by the tracker, whether optical or otherwise, about one or more tracking markers and usable for determining the location of the markers and their orientation and movement continually in ‘real time’ during a procedure. The term “vectorized” is used in this specification to describe fiducial keys and tracking markers that are at least one of shaped and marked, or have a portion that is one of shaped and marked, so as to make their orientation in three dimensions uniquely determinable from their appearance in a scan or in image information. If their three-dimensional orientation is determinable, then their three-dimensional location is also known. Fiducial keys and tracking markers disclosed in this specification may have rotationally asymmetric shapes or bear rotationally asymmetric patterns of markings to render them vectorized.

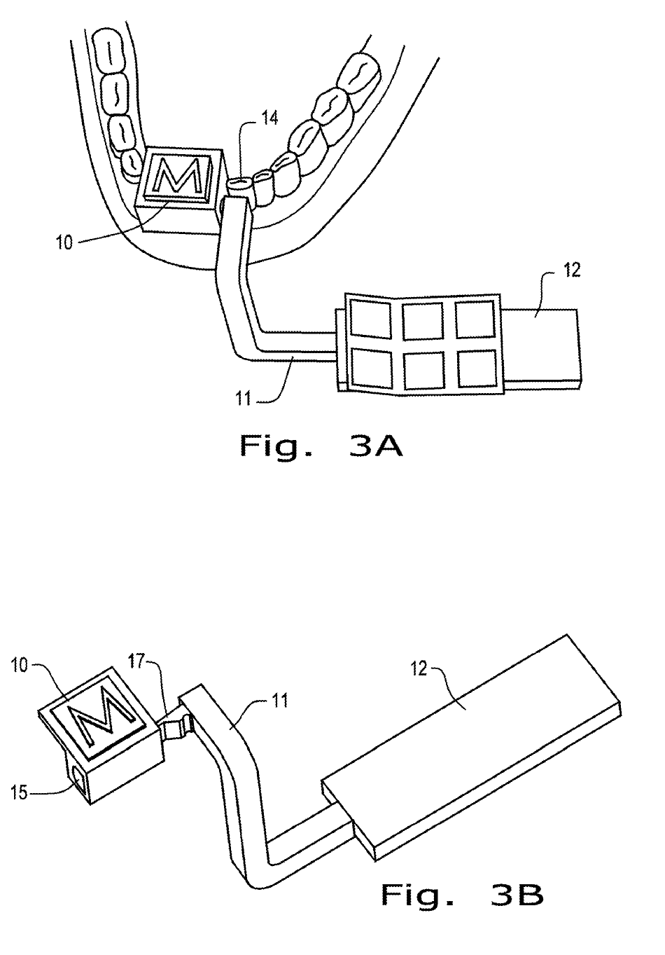

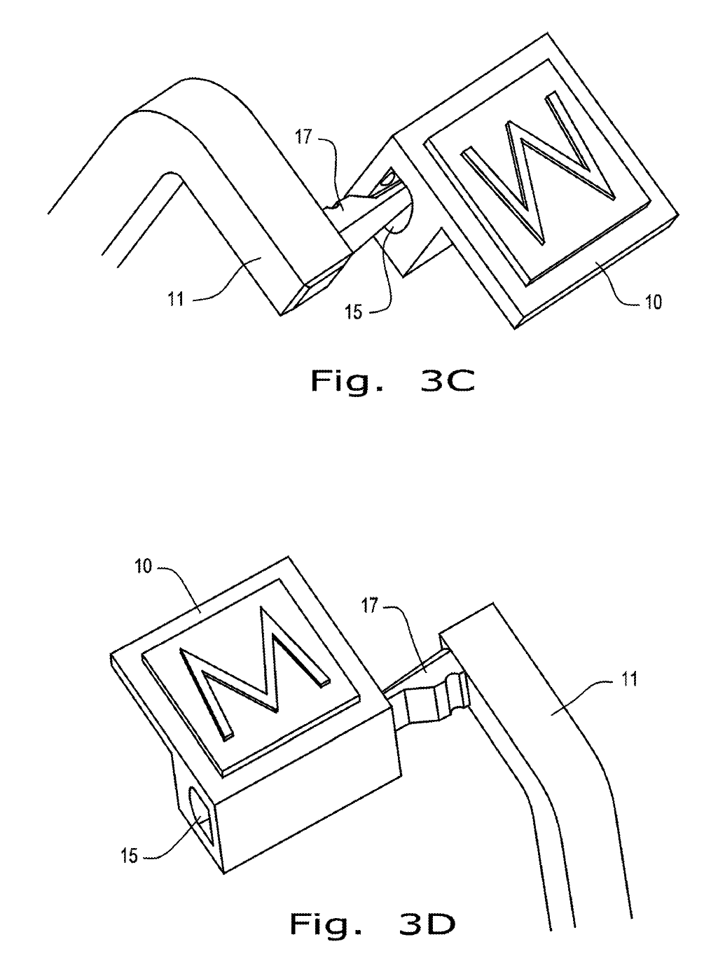

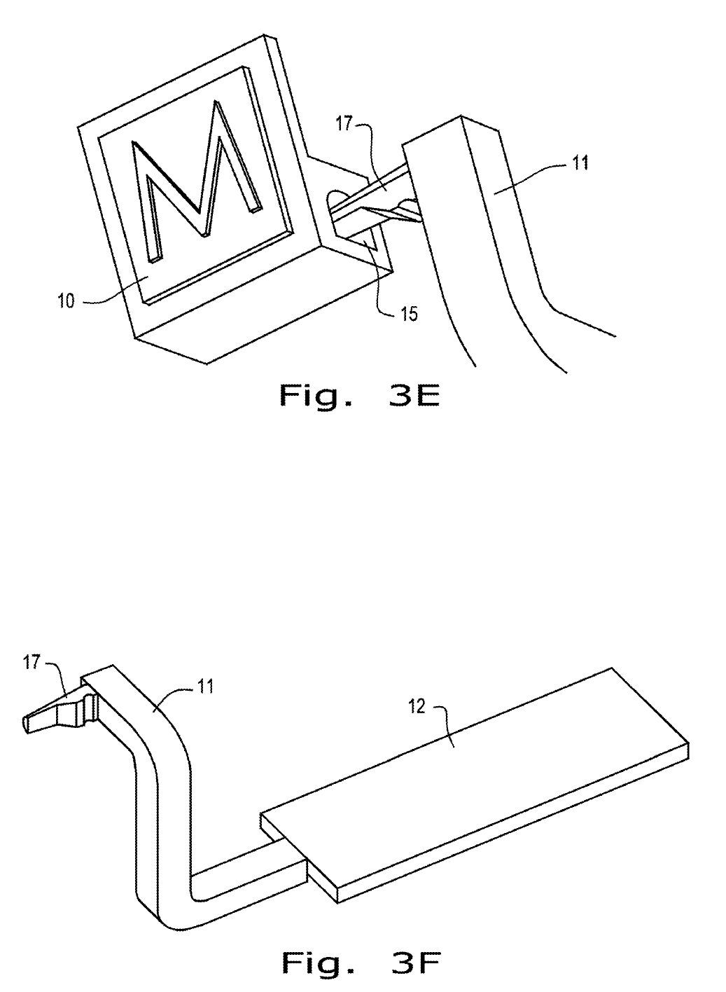

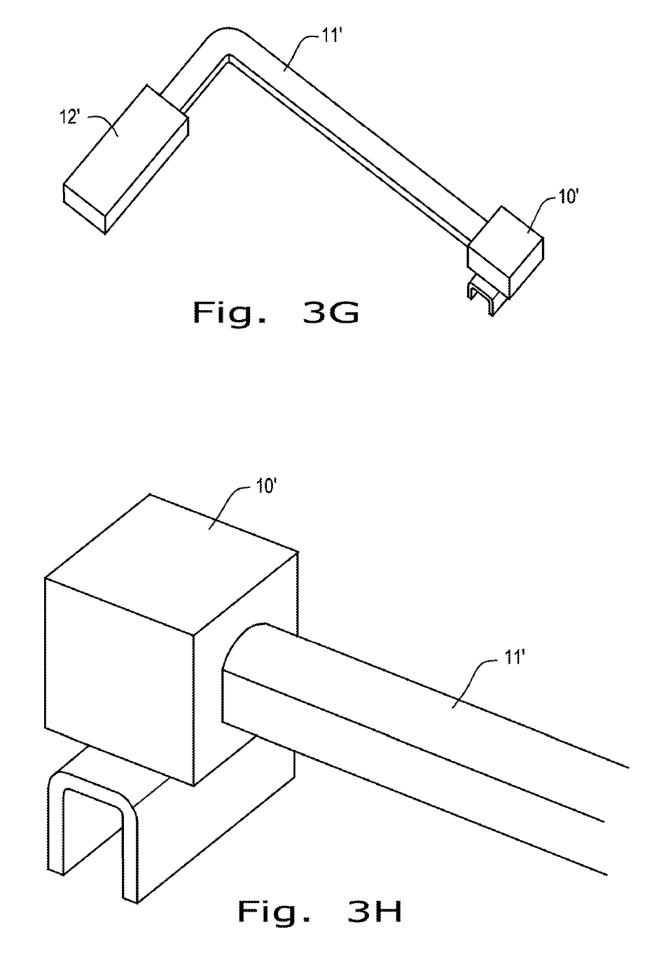

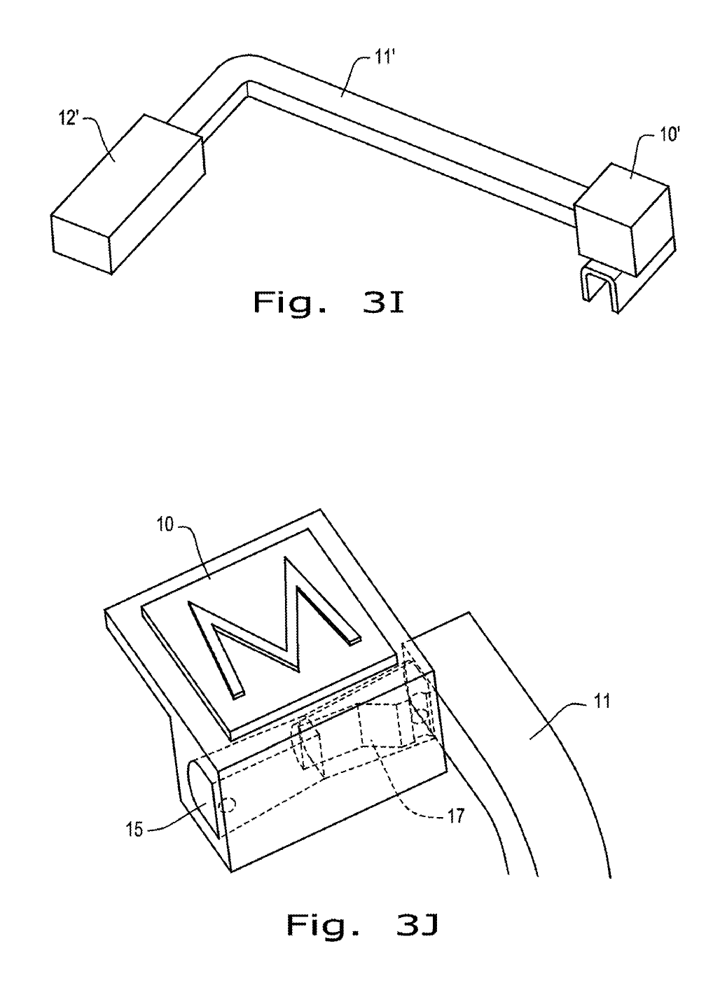

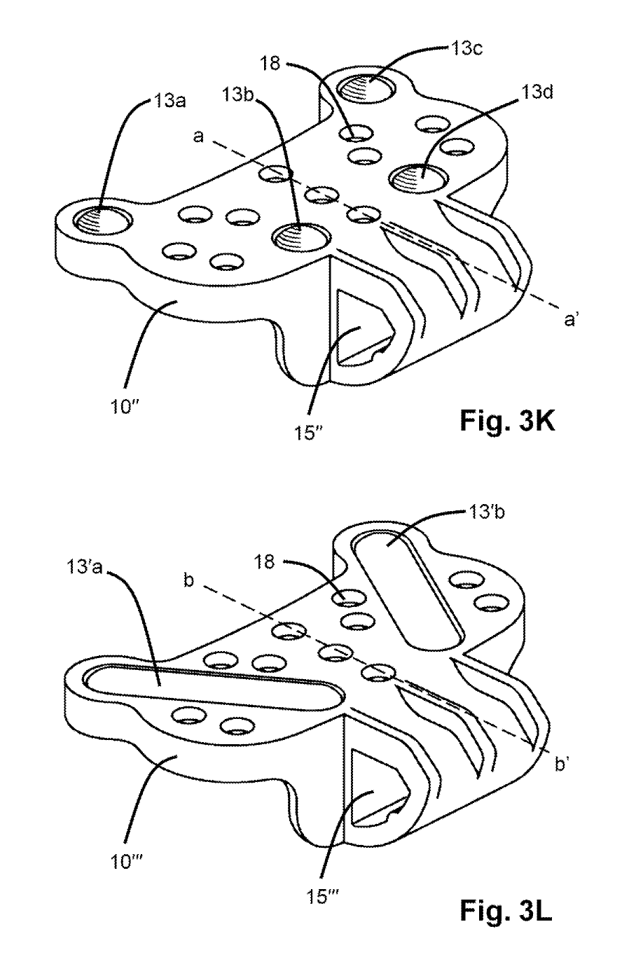

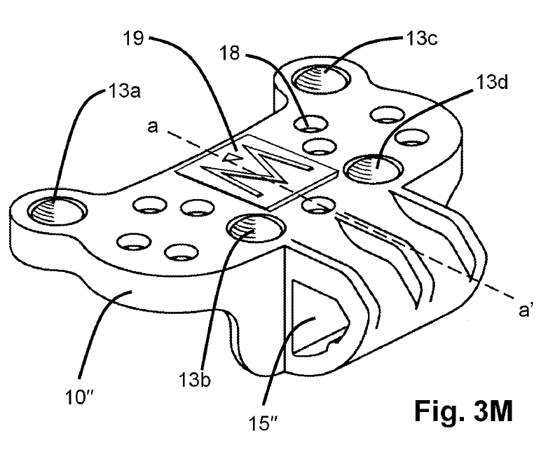

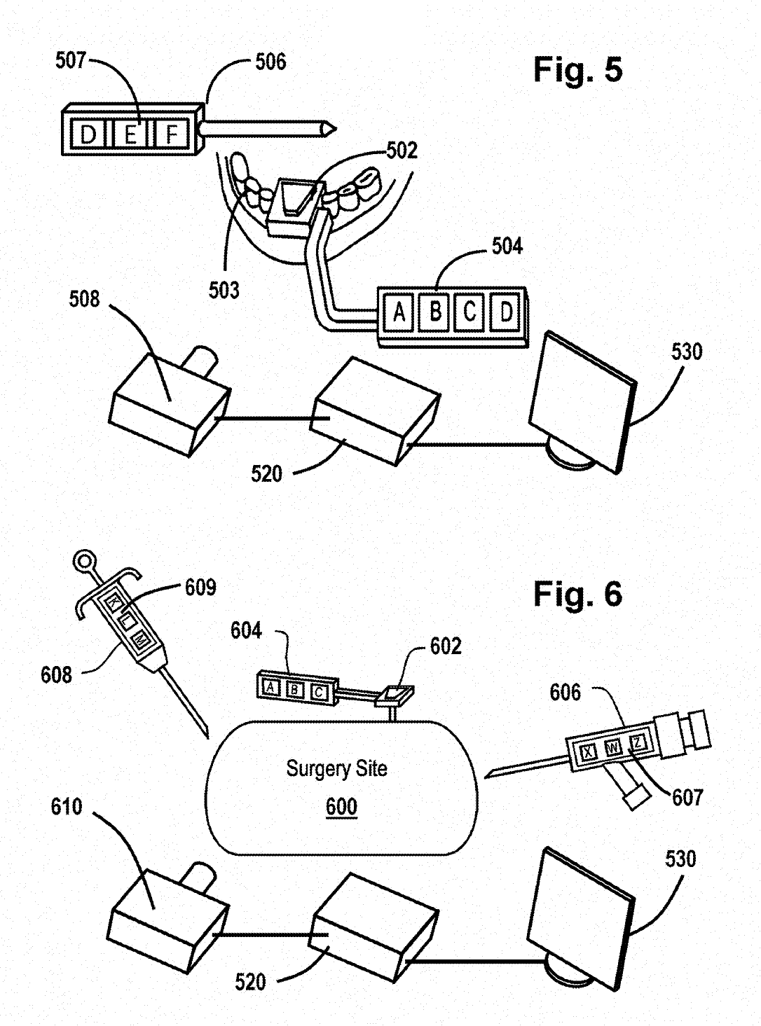

All vectorized tracking markers employed in the present invention (for example 504, 507, 607 and 609 of FIG. 5 and FIG. 6) may be passive. The term “passive” is used in the present specification to describe markers that do not rely on any own electronic, electrical, optoelectronic, optical, magnetic, wireless, inductive, or other active signaling function or on any incorporated electronic circuit, whether powered or unpowered, to be identified, located, or tracked. The term “own active signaling” is used in this specification to describe a signal that is temporally modulated by, on, or within the tracking marker. The tracking markers do not rely on motion, location, or orientation sensing devices, whether powered or unpowered, to be tracked. They cannot sense their own motion, location, or orientation, nor have they any ability to actively communicate. They bear distinctive markings and/or have distinctive shapes that allow them to be identified, located, and tracked in three dimensions by a separate tracker such as, for example, tracker 610 of FIG. 6, both in their location and in their orientation. In some embodiments, the tracker may be an optical tracker, more particularly, a non-stereo optical tracker. Any one or more of identification, location, and tracking of the markers is solely on the basis of their distinctive markings and/or distinctive shapes, or on the basis of the distinctive markings and/or distinctive shape of a portion of a tracker being tracked. All fiducial references employed in the present invention may also be passive. This specifically includes fiducial references 502 and 602 of FIG. 5 and FIG. 6 and fiducial references 10, 10′, 10″ and 10′″ in FIGS. 3A to 3M.

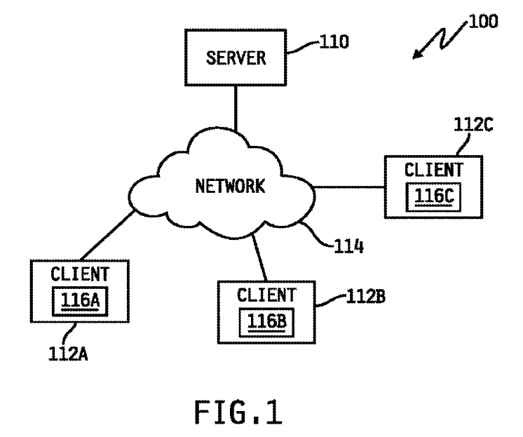

FIG. 1 is a high-level block diagram of a computing environment 100 according to one embodiment. FIG. 1 illustrates server 110 and three clients 112 connected by network 114. Only three clients 112 are shown in FIG. 1 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

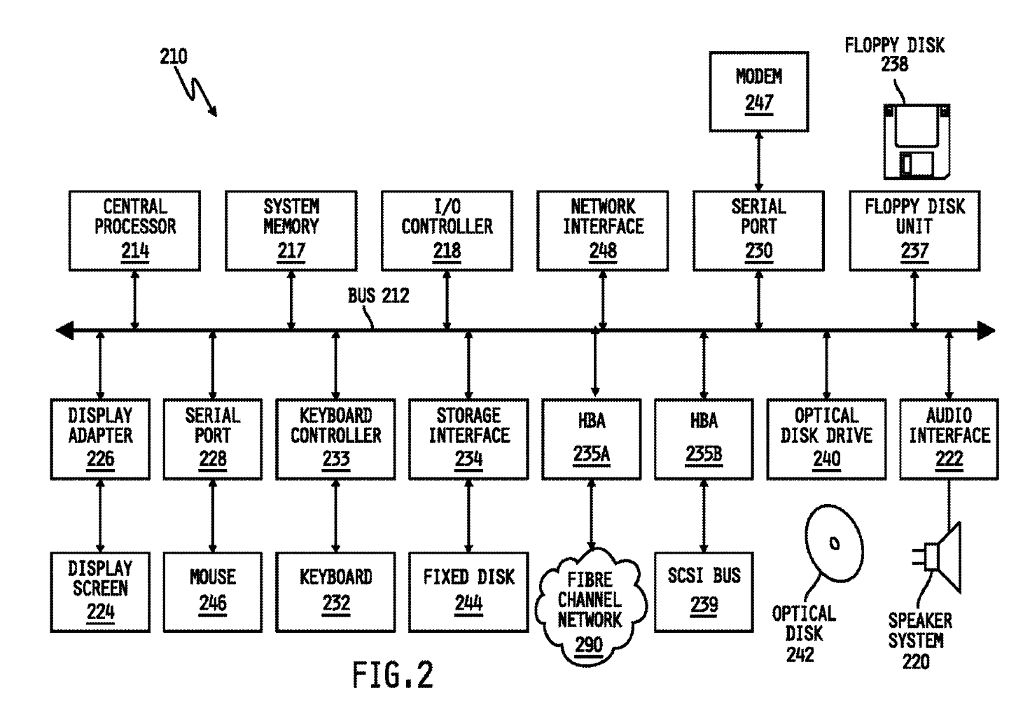

FIG. 2 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238 (or other suitable portable storage, e.g., a memory stick or card), host bus adapter (HBA) interface card 235A operative to connect with Fiber Channel network 290, host bus adapter (HBA) interface card 235B operative to connect to SCSI bus 239, and optical disk drive 240 operative to receive optical disk 242. Also included are mouse 246 (or other point-and-click device coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS), which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium. Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an Internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

Many other devices or subsystems (not shown) may be connected in a similar manner (e. g., document scanners, digital cameras and so on), including the hardware components of FIGS. 5 and 6, which alternatively may be in communication with associated computational resources through local, wide-area, or wireless networks or communications systems. Thus, while the disclosure may generally discuss an embodiment where the hardware components are directly connected to computing resources, one of ordinary skill in this area recognizes that such hardware may be remotely connected with computing resources. Conversely, all of the devices shown in FIG. 2 need not be present to practice the present disclosure. Devices and subsystems may be interconnected in different ways from that shown in FIG. 2. Operation of a computer system such as that shown in FIG. 2 is readily known in the art and is not discussed in detail in this application. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Wash.), WINDOWS® (WINDOWS is a registered trademark of Microsoft Corporation of Redmond, Wash.), OS/2® (OS/2 is a registered trademark of International Business Machines Corporation of Armonk, New York), UN]X® (UNLX is a registered trademark of X/Open Company Limited of Reading, United Kingdom), Linux® (Linux is a registered trademark of Linus Torvalds of Portland, Oreg.), or other known or developed operating system.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above-described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modification to the first signal, whether due to circuit limitations or due to passage through other circuit elements which do not change the informational and/or final functional aspect of the first signal.

The present invention relates to embodiments of surgical hardware and software monitoring systems and methods which allow for surgical planning while the patient is available for surgery, for example while the patient is being prepared for surgery so that the system may model the surgical site. The system uses a particularly configured piece of hardware, namely a vectorized fiducial reference, represented as fiducial key 10 in FIG. 3A, to orient vectorized tracking marker 12 of the monitoring system with regard to the critical area of the surgery. Single fiducial key 10 is attached to a location near the intended surgical area, in the exemplary embodiment of the dental surgical area of FIG. 3A, fiducial key 10 is attached to a dental splint 14. Vectorized tracking marker 12 may be connected to fiducial key 10 by tracking pole 11. In embodiments in which the fiducial reference is directly visible to a suitable tracker (see for example FIG. 5 and FIG. 6) that acquires image information about the surgical site, a tracking marker may be attached directly to the fiducial reference, being fiducial key 10 in the present embodiment. The tracker may be a non-stereo optical tracker. For example, in a dental surgery, dental tracking marker 14 may be used to securely locate fiducial 10 near the surgical area. Single fiducial key 10 may be used as a point of reference, or a fiducial, for the further image processing of data acquired from tracking marker 12 by the tracker. In this arrangement, the fiducial key or reference 10 is scanned not by the tracker, which may for example be an optical tracker, but by a suitable scanning means, which may for example be an X-ray system, CAT scan system, or MRI system as per the definition of “scan” above. In some applications, fiducial key 10 may be disposed in a location or in such orientation as to be at least in part non-visible to the tracker of the system.

In other embodiments additional vectorized tracking markers 12 may be attached to items independent of fiducial key 10 and any of its associated tracking poles 11 or tracking markers 12. This allows the independent items to be tracked by the tracker.

In a further embodiment at least one of the items or instruments near the surgical site may optionally have a tracker attached to function as tracker for the monitoring system of the invention and to thereby sense the orientation and the position of tracking marker 12 and of any other additional vectorized tracking markers relative to the scan data of the surgical area. By way of example, the tracker attached to an instrument may be a miniature digital camera and it may be attached, for example, to a dentist's drill. Any other vectorized markers to be tracked by the tracker attached to the item or instrument must be within the field of view of the tracker.

Using the dental surgery example, the patient is scanned to obtain an initial scan of the surgical site. The particular configuration of single fiducial key 10 allows computer software stored in memory and executed in a suitable controller, for example processor 214 and memory 217 of computer 210 of FIG. 2, to recognize its relative position within the surgical site from the scan data, so that further observations may be made with reference to both the location and orientation of fiducial key 10. In some embodiments, the fiducial reference includes a marking that is apparent, for example, as a recognizable identifying symbol when scanned. In other embodiments, the fiducial reference includes a shape that is distinct in the sense that the body apparent on the scan has an asymmetrical form allowing the front, rear, upper, and lower, and left/right defined surfaces that may be unambiguously determined from the analysis of the scan, thereby to allow the determination not only of the location of the fiducial reference, but also of its orientation. That is, the shape and/or markings of the fiducial reference render it vectorized. The marking and/or shape of fiducial key 10 allows it to be used as the single and only fiducial key employed in the surgical hardware and software monitoring system. By comparison, prior art systems typically rely on a plurality of fiducials. Hence, in the present invention, while the tracker may track several vectorized tracking markers within the monitoring system, only a single vectorized fiducial reference or key 10 of known shape or marking is required. By way of example, FIG. 5, later discussed in more detail, shows vectorized markers 504 and 507 tracked by tracker 508, but there is only one vectorized fiducial reference or key 502 in the system. FIG. 6 similarly shows three vectorized markers 604, 607, and 609 being tracked by tracker 610, while there is only a single vectorized fiducial reference or key 602 in the system.

In addition, the computer software may create a coordinate system for organizing objects in the scan, such as teeth, jaw bone, skin and gum tissue, other surgical instruments, etc. The coordinate system relates the images on the scan to the space around the fiducial and locates the instruments bearing markers both by orientation and position. The model generated by the monitoring system may then be used to check boundary conditions, and in conjunction with the tracker display the arrangement in real time on a suitable display, for example display 224 of FIG. 2.

In one embodiment, the computer system has a predetermined knowledge of the physical configuration of single fiducial key 10 and examines slices/sections of the scan to locate fiducial key 10. Locating of fiducial key 10 may be on the basis of its distinct shape, or on the basis of distinctive identifying and orienting markings upon the fiducial key or on attachments to the fiducial key 10 such as tracking marker 12. Fiducial key 10 may be rendered distinctly visible in the scans through higher imaging contrast by the employ of radio-opaque materials or high-density materials in the construction of the fiducial key 10. In other embodiments the material of the distinctive identifying and orienting markings may be created using suitable high density or radio-opaque inks or materials.

Once fiducial key 10 is identified, the location and orientation of the fiducial key 10 is determined from the scan segments, and a point within fiducial key 10 is assigned as the center of the coordinate system. The point so chosen may be chosen arbitrarily, or the choice may be based on some useful criterion. A model is then derived in the form of a transformation matrix to relate the fiducial system, being fiducial key 10 in one particular embodiment, to the coordinate system of the surgical site. The resulting virtual construct may be used by surgical procedure planning software for virtual modeling of the contemplated procedure, and may alternatively be used by instrumentation software for the configuration of the instrument, for providing imaging assistance for surgical software, and/or for plotting trajectories for the conduct of the surgical procedure.

In some embodiments, the monitoring hardware includes a tracking attachment to the fiducial reference. In the embodiment pertaining to dental surgery the tracking attachment to fiducial key 10 is tracking marker 12, which is attached to fiducial key 10 via tracking pole 11. Tracking marker 12 may have a particular identifying pattern. The pattern may be a rotationally asymmetric pattern. The trackable attachment, for example tracking marker 12, and even associated tracking pole 11 may have known configurations so that observational data from tracking pole 11 and/or tracking marker 12 may be precisely mapped to the coordinate system, and thus progress of the surgical procedure may be monitored and recorded. For example, as particularly shown in FIG. 3J, fiducial key 10 may have hole 15 in a predetermined location specially adapted for engagement with insert 17 of tracking pole 11. In such an arrangement, for example, tracking poles 11 may be attached with a low force push into hole 15 of fiducial key 10, and an audible haptic notification may thus be given upon successful completion of the attachment.

It is further possible to reorient the tracking pole during a surgical procedure. Such reorientation may be in order to change the location of the procedure, for example where a dental surgery deals with teeth on the opposite side of the mouth, where a surgeon switches hands, and/or where a second surgeon performs a portion of the procedure. For example, the movement of the tracking pole may trigger a re-registration of the tracking pole with relation to the coordinate system, so that the locations may be accordingly adjusted. Such a re-registration may be automatically initiated when, for example in the case of the dental surgery embodiment, tracking pole 11 with its attached tracking marker 12 are removed from hole 15 of fiducial key 10 and another tracking marker with its associated tracking pole is connected to an alternative hole on fiducial key 10. Additionally, boundary conditions may be implemented in the software so that the user is notified when observational data approaches and/or enters the boundary areas.

The tracker of the system may comprise a single optical imager obtaining a two-dimensional image of the site being monitored. The system and method described in the present specification allow three-dimensional locations and orientations of tracking markers to be obtained using non-stereo-pair two-dimensional imagery. In some embodiments more than one imager may be employed as tracker, but the image information required and employed is nevertheless two-dimensional. Therefore the two imagers may merely be employed to secure different perspective views of the site, each imager rendering a two-dimensional image that is not part of a stereo pair. This does not exclude the employment of stereo-imagers in obtaining the image information about the site, but the system and method are not reliant on stereo imagery of the site.

In a further embodiment, the vectorized tracking markers may specifically have a three-dimensional shape. Suitable three-dimensional shapes bearing identifying patterns may include, without limitation, a segment of an ellipsoid surface and a segment of a cylindrical surface. In general, suitable three-dimensional shapes are shapes that are mathematically describable by simple functions.

The term “identifiably unique” is employed in the present specification to describe a pattern that is distinct from patterns on any other tracking markers employed with the system and may be uniquely identified with a particular tracking marker for the purposes of identifying the marker, both when it is used alone and when used in conjunction with other pattern-bearing tracking markers. The term “pattern reference point” is employed in the present specification to describe a consistently identifiable point within the rotationally asymmetric pattern on a tracking marker that may be employed in determining a coordinate system for purposes of describing the three-dimensional locations and orientations of elements of the tracking system. The rotationally asymmetric pattern may comprise pattern elements having contrast with respect to a background.

In a further embodiment of the system utilizing the invention, a surgical instrument or implement, herein termed a “hand piece” (see FIGS. 5 and 6), may also have a particular configuration that may be located and tracked in the coordinate system and may have suitable tracking markers as described herein. A boundary condition may be set up to indicate a potential collision with virtual material, so that when the hand piece is sensed to approach the boundary condition an indication may appear on a screen, or an alarm sound. Further, target boundary conditions may be set up to indicate the desired surgical area, so that when the trajectory of the hand piece is trending outside the target area an indication may appear on screen or an alarm sound indicating that the hand piece is deviating from its desired path.

An alternative embodiment of some hardware components are shown in FIGS. 3G-I. Vectorized fiducial key 10′ has connection elements with suitable connecting portions to allow tracking pole 11′ to position tracking marker 12′ relative to the surgical site. Conceptually, fiducial key 10′ serves as an anchor for pole 11′ and tracking marker 12′ in much the same way as the earlier embodiment, although it has a distinct shape. The software of the monitoring system is pre-programmed with the configuration of each particularly identified fiducial key, tracking pole, and tracking marker, so that the location calculations are only changed according to the changed configuration parameters.

The materials of the hardware components may vary according to regulatory requirements and practical considerations. Generally, the key or fiducial component is made of generally radio opaque material such that it does not produce noise for the scan, yet creates recognizable contrast on the scanned image so that any identifying pattern associated with it may be recognized. In addition, because it is generally located on the patient, the material should be lightweight and suitable for connection to an apparatus on the patient. For example, in the dental surgery example, the materials of the fiducial key must be suitable for connection to a plastic splint and suitable for connection to a tracking pole. In the surgical example the materials of the fiducial key may be suitable for attachment to the skin or other particular tissue of a patient.

The vectorized tracking markers may be clearly identified by employing, for example without limitation, high contrast pattern engraving. The materials of the tracking markers are chosen to be capable of resisting damage in autoclave processes and are compatible with rigid, repeatable, and quick connection to a connector structure. The tracking markers and associated tracking poles have the ability to be accommodated at different locations for different surgery locations, and, like the fiducial keys, they should also be relatively lightweight as they will often be resting on or against the patient. The tracking poles must similarly be compatible with autoclave processes and have connectors of a form shared among tracking poles.

FIG. 3K, shows a multi-material fiducial reference 10″ comprising at least two distinct materials. The first material is that of the structural body of fiducial reference 10″ and the second material is that of scan-detectable elements 13a, 13b, 13c, and 13d embedded in the structural body of fiducial reference 10″. The first material is chosen for its biocompatibility at the surgical site, its dimensional rigidity, and its formability. The body of fiducial reference 10″ may be formed via any one of a variety of methods, including but not limited to casting, injection molding or three-dimensional printing. The term “multi-material fiducial” is used in the present specification to describe a fiducial comprising at least the two materials described above and below. In some embodiments, the multi-material fiducial may comprise further materials serving further functions.

The second material may be chosen for its ability to be clearly imaged during a scan of the type described above. In this respect it should be noted that medical scanning systems are often optimized in terms of, for example, their scan contrast, scan brightness and scan gamma in detecting biological materials such as human bone. Fiducials are therefore typically made of radio-opaque materials capable of producing suitable contrast during a scan optimized for biological materials. Suitable materials as choice for the second material in FIG. 3K include, but are not limited to, a metal or metallic-oxide ceramic, for example stainless steel, titanium, aluminum oxide, zirconium oxide, and silicon nitride. Forming the body of fiducial reference 10″ and scan-detectable elements 13a, 13b, 13c, and 13d from different materials allows the choice of the two materials to be separately optimized for their respective roles. It also changes relative to the prior art the way in which fiducial reference 10″ may be employed during surgery or surgical planning. This is discussed in more detail below.

While the physical outline of fiducial reference 10″ in FIG. 3K may in some implementations have 180° rotational symmetry about an axis parallel to broken line a-a′, scan-detectable elements 13a, 13b, 13c, and 13d are arranged within fiducial reference 10″ to have zero three-dimensional rotational symmetry. That is, there exists no axis about which fiducial reference 10″ may be rotated by less than 360° to obtain the same mutual three-dimensional juxtaposition of scan-detectable elements 13a, 13b, 13c, and 13d. This three-dimensional rotational asymmetry of the implant arrangement ensures that both the position and the three-dimensional orientation of fiducial reference 10″, also known jointly as its “pose”, may be uniquely determined from the scan data obtained of the surgical site with fiducial reference 10″ attached to the surgical site at the time of the scan. In FIG. 3K, four scan sensitive implants are employed, but in a more general implementation only three point, spherical, or ball elements are required to absolutely identify the three-dimensional location and orientation of fiducial reference 10″ from the scan data.

As explained in the foregoing sections of this specification, suitable tracking attachments may be attached to reference 10″ via hole 15″. In the embodiment pertaining to dental surgery the tracking attachment to fiducial key 10″ is tracking marker 12, which is attachable to fiducial key 10″ via a suitable tracking pole, for example tracking pole 11. Holes 18 in fiducial reference 10″ are employed to provide more adhesion for the dental putty employed in fitting fiducial reference 10″ to the teeth of the patient.

In FIG. 3L, fiducial reference 10′″ may comprise, instead of the point, spherical, or ball elements of FIG. 3K, at least two rod-shaped scan-detectable elements 13′a and 13′b made of materials of the same characteristics and requirements as scan-detectable elements 13a, 13b, 13c, and 13d of FIG. 3K. In this case also, the material of the fiducial reference 10′″ is chosen for its biocompatibility at the surgical site, its dimensional rigidity, and its formability. The body of fiducial reference 10″ may be formed via any one of a variety of methods, including but not limited to injection molding. Suitable tracking attachments may be attached to reference 10′″ via hole 15′″. In the embodiment pertaining to dental surgery the tracking attachment to fiducial key 10′″ is tracking marker 12, which is attachable to fiducial key 10′″ via a suitable tracking pole, for example tracking pole 11.

As in the case of FIG. 3K, even though the outline of the body of fiducial reference 10′″ of FIG. 3L may very well have 180° rotational symmetry about an axis parallel to broken line b-b′, scan-detectable elements 13′a and 13′b are arranged within fiducial reference 10′″ to have zero three-dimensional rotational symmetry. That is, there exists no axis about which fiducial reference 10′″ may be rotated by less than 360° to obtain the same mutual juxtaposition of scan-detectable elements 13′a and 13′b. To this end, elements 13′a and 13′b may be, for example without limitation, of different length, or may be oriented at different angles with respect to line b-b′. More than two rod-shaped scan sensitive elements may be employed, but a minimum of two rod-shaped scan sensitive elements is required in order to obtain a unique three-dimensional location and orientation of fiducial reference 10′″ from scan data. Holes 18 in fiducial reference 10′″ are employed to provide more adhesion for the dental putty employed in fitting fiducial reference 10′″ to the teeth of the patient.

In embodiments based on the principles and elements elucidated in FIGS. 3K and 3L, a fiducial reference for use in planning and tracking surgery at a surgical site comprises: a fiducial reference formed of a biocompatible material having dimensional rigidity; and a rigidly embedded three-dimensionally asymmetric scan-detectable element, the scan-scan-detectable element identifiable in a scan of the surgical site. The scan-detectable element may comprise a plurality of individual elements rigidly arranged with respect to one another to render the scan-detectable element three-dimensionally asymmetric. All of the plurality of elements may be identical and the three-dimensionally asymmetric characteristic of the scan-detectable element may be due entirely to the mutual arrangement of the plurality of individual elements. Suitable materials as choice for the scan-detectable element include, but are not limited to, a metal or metallic-oxide ceramic, for example stainless steel, titanium, aluminum oxide, zirconium oxide, and silicon nitride. The scan-detectable element may be wholly contained within the body of the biocompatible fiducial reference such as to not be visible to the human eye. In some embodiments, for example without limitation those shown in FIGS. 3K and 3L, scan-detectable element may be partially contained within the body of the biocompatible fiducial reference.

In the multi-material embodiments based on FIGS. 3K and 3L and as described in the paragraph immediately above, the fact that the scan-detectable element is rigidly embedded in the fiducial reference allows the pose of the scan-detectable element to be known with great precision and accuracy with respect to any tracking marker attached to the fiducial reference. Knowledge of the pose of the tracking marker therefore renders the pose of the fiducial and its scan-detectable element known also. The accurate and precise pose of the scan-detectable element with respect to the fiducial reference is determined during the manufacture of the fiducial reference. This mutual positioning and orienting is required to be done to an accuracy and precision that is in keeping with the demands of the surgery for which the system is provided. In the present specification we refer to this appropriate level of accuracy and precision using the phrase “surgical precision and accuracy”. It is the lack of surgical precision and accuracy that forces users of prior art tracking systems to calibrate any fiducials and/or tracking markers prior to use. To the extent that the fiducial references of the present invention are used without requiring any calibration before use, both “surgical precision” and “surgical accuracy” are required in the manufacture of the bi-material fiducials based on FIGS. 3K and 3L and as described in the paragraph immediately above. This requires the surgically precise and surgically accurate positioning of the scan-identifiable element during the forming of the fiducials as part of the manufacturing of the fiducials. The forming process for the body of the fiducials may be, for example without limitation, casting, injection molding or three-dimensional printing.

The term “accuracy” is employed in this specification to describe the closeness of the placement of a scan-detectable element to its intended placement. The term “precision” is used to describe the repeatability of the placement of a scan-detectable element. Due to the accuracy and precision with which scan-detectable elements 13a, 13b, 13c, and 13d of fiducial key 10″ in FIG. 3K are positioned during manufacture of fiducial key 10″, fiducial key 10″ may be used as a user-calibration-free fiducial key. The adjective term “user-calibration-free” is employed in the present specification to describe fiducial references, and tracking markers rigidly attached to them, that require no calibration by the user. The “pose” of the fiducial references may be determined to a “surgical precision and accuracy” by the tracking system of the present invention so that any spatial calibration of the fiducials or markers attached to them during use is obviated. As the pose of the embedded scan-detectable element with respect to the fiducial reference is known with the same or better accuracy and precision, and the pose of the scan-detectable element with respect to the surgical site is known from a suitable prior scan of the surgical site, tracking of the fiducial reference, either directly or via tracking of a rigidly and removably attached tracking marker, in the image information suffices to allow the tracking system to provide the pose of the surgical site with “surgical precision and accuracy”.

In exemplary tracking systems made by the inventors, the surgical accuracy that enables the user-calibration-free aspect of the tracking system, tracking markers and fiducial reference is achieved by positioning the scan-detectable elements within the fiducial reference during the manufacture of the latter to an accuracy of better than 150 microns. That is, the placement of any point on or in a scan-detectable element is within +/−150 microns of its specified placement position. The precision at 150_micron accuracy is 100%. That is, every point on or in every scan-detectable element is within 150 microns of its specified placement position.

In dental surgery, the relationship between the accuracy of the placement of the scan-detectable elements and the accuracy of the surgery is determined by the ratio between the distance from one of the scan-detectable elements to the rearmost teeth, on the one hand, to the shortest distance between scan-detectable elements in the fiducial reference on the other hand. The resulting ratio, which refer to as a “lever ratio” in the present specification, creates by a lever action an accuracy that is on the order of six times worse at the third lower adult human molar as compared with the accuracy at the fiducial attached to one of the lower adult human central incisors. This implies that a +/−150 micron placement accuracy of the scan-detectable element in the fiducial reference at the lower central incisor results in an accuracy of +/−900 microns at the third lower molar. This accuracy is generally deemed suitable for use in dental surgery. Other sources of inaccuracies may however be compounded with the inaccuracy in placement of the scan-detectable elements in the fiducial reference to render the overall accuracy of the system as whole outside the limit of +/−

In further, more developed exemplary tracking systems, the placement accuracy is better than +/−80 microns with a 100% precision. This leads to an accuracy of +/−480 microns at the third lower molar. This is an accuracy that is generally safely acceptable in surgical practice.

In yet further exemplary tracking systems, the placement accuracy is better than +/−40 microns with a 100% precision. This leads to an accuracy of +/−240 microns at the third lower molar. This is an accuracy that is generally deemed to be the best that may be achieved by hand, making the system of the present invention comparable in accuracy to the very best that may be achieved by hand.

In yet further exemplary tracking systems, the placement accuracy is better than +/−16 microns with a 100% precision. This leads to an accuracy of +/−96 microns at the third lower molar. This is an accuracy that is generally deemed to represent negligible deviation so that any inaccuracy introduced by the placement of the scan-detectable elements in the fiducial reference effectively disappears in comparison to other sources of inaccuracy.

In the present specification, the four levels of accuracy compatible with human surgery described above may be attained with 100% precision for the fiducial reference of the present invention by the methods described below.

FIG. 3M shows a further embodiment, wherein a uniquely identifiable marker is disposed directly on a fiducial reference. By way of non-limiting example, we employ for this purpose the fiducial reference of FIG. 3K, on which is disposed trackable marker 19 comprising an identifiable pattern. Fiducial reference 10″ and marker 19 may not be mutually drawn to scale in FIG. 3M. In this embodiment the pattern of marker 19 is required to be at least in part visible to the tracker of the system, for example tracker 520 of FIG. 5 and FIG. 6 discussed in more detail later. The tracker may be an optical tracker and more specifically, a non-stereo optical tracker. As with other tracking markers described in this specification, marker 19 bears an optically identifiable pattern with no rotational symmetry consisting of areas having suitable contrast with respect to a background. The pattern may be discernible by the tracker of the system in the visible, infrared or ultra-violet regions of the spectrum. This allows the pose of marker 19, and thereby the pose of fiducial reference 10″ to be uniquely determined from the image information obtained by the tracker of the system. This arrangement does not require a separate tracking pole or separate tracking marker to be attached to fiducial reference 10″ via hole 15″. It may be employed at surgical sites where tracking marker 19 on fiducial reference 10″ is at least in part visible to the tracker, a portion of the pattern on marker 19 being adequate to determine the pose of fiducial reference 10″.

The embodiments shown in FIGS. 3K, 3L and 3M show that a tracking marker may be rigidly attached to the fiducial reference at a predetermined location in a predetermined three-dimensional orientation with respect to the fiducial reference, irrespective of whether the marker is directly on the fiducial reference or whether it is attached via a mounting hole, for example mounting hole 15″ or 15′″.

To achieve the abovementioned placement accuracies of the scan-detectable elements within the fiducial references, the fiducial reference is formed around the scan-detectable elements while the scan-detectable elements are held rigidly in place. In the case of injection molding, the pre-made scan-detectable elements may be held rigidly in position to the abovementioned accuracies within the mold while the fiducial reference is injection-molded around them. One non-limiting example of a method for holding the scan-detectable elements rigidly in place is by using at least one pin, in some embodiments multiple pins. In those cases where the scan-detectable elements are wholly surrounded by the material of the fiducial reference, more than one injection molding step may be required. In the first step, the scan-detectable elements are embedded but not wholly surrounded. In a second injection step the rest of the fiducial reference is formed while the scan-detectable elements remain rigidly held in place by the injection molded material of the first injection molding step. Placement accuracies as good as +/−16 microns may be obtained with 100% precision for the scan-detectable elements within the fiducial references by this method of manufacture, as one of skill in this art of manufacturing would recognize. We return later to this method.

The tracker employed in tracking the fiducial keys, tracking poles and tracking markers may be capable of tracking with suitable accuracy objects of a size of the order of 1.5 square centimeters. While the tracker is generally connected by wire to a computing device to read the sensory input, it may optionally have wireless connectivity to transmit the sensory data to a computing device. The tracker may be a non-stereo optical tracker.

In embodiments that additionally employ a trackable piece of instrumentation, such as a hand piece, vectorized tracking markers attached to such a trackable piece of instrumentation may also be light-weight; capable of operating in a 3 object array with 90 degrees relationship; optionally having a high contrast pattern engraved or attached and a rigid, quick mounting mechanism to a standard hand piece.

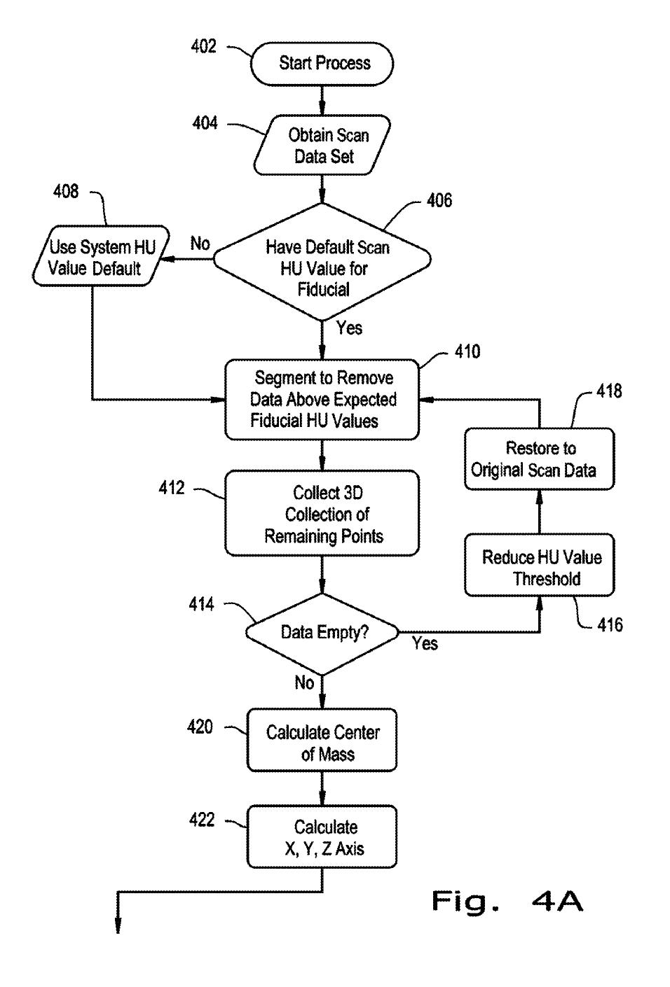

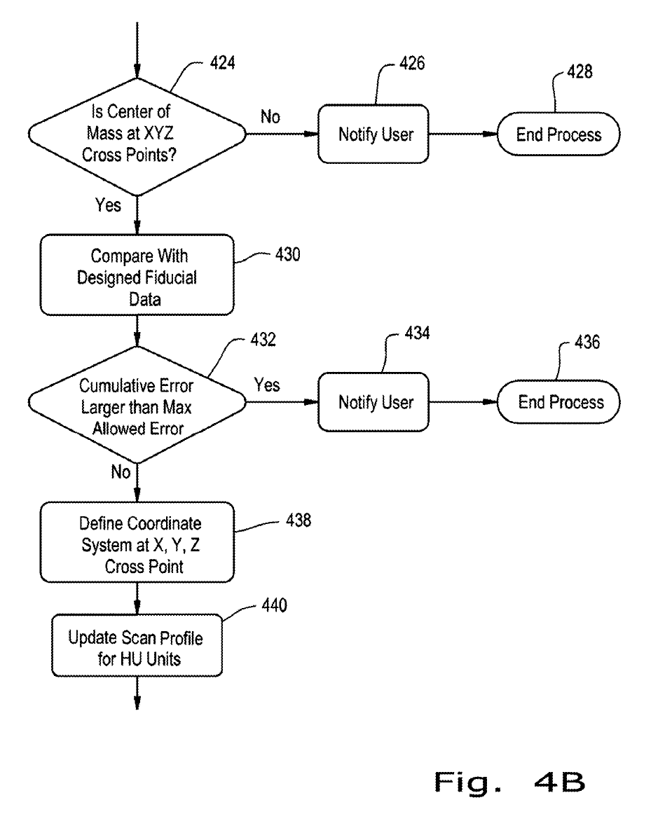

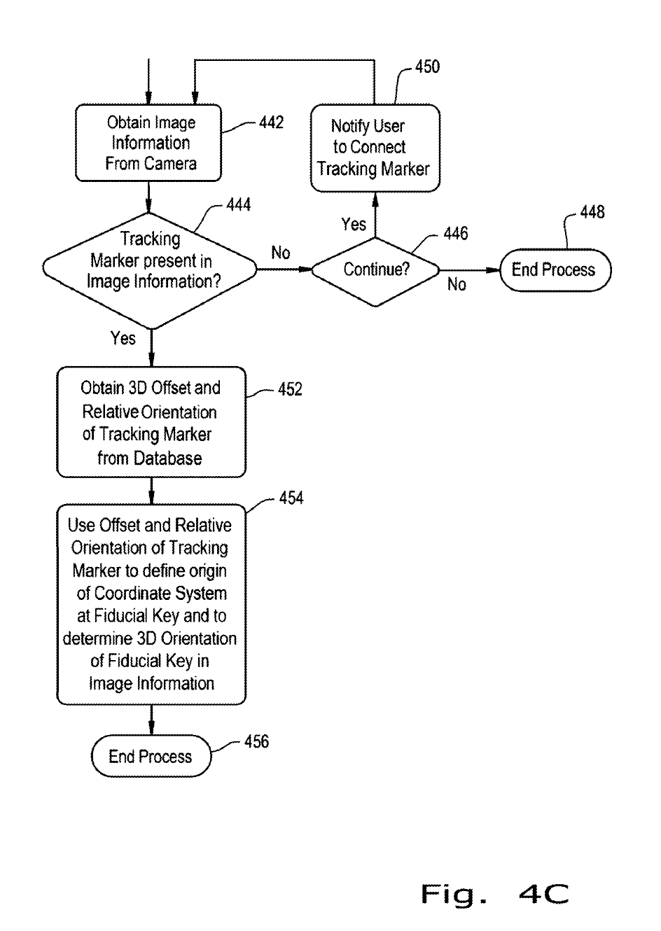

In another aspect there is presented an automatic registration method for tracking surgical activity, as illustrated in FIGS. 4A-C. FIG. 4A and FIG. 4B together present, without limitation, a flowchart of one method for determining the three-dimensional location and orientation of the fiducial reference from scan data. FIG. 4C presents a flow chart of a method for confirming the presence of a suitable tracking marker in image information obtained by the tracker and determining the three-dimensional location and orientation of the fiducial reference based on the image information.

Once the process starts [402], as described in FIGS. 4A and 4B, the system obtains [404] a scan data set from, for example, a CT scanner and checks [at 406] for a default CT scan Hounsfield unit (HU) value for the vectorized fiducial which may or may not have been provided with the scan based on a knowledge of the fiducial and the particular scanner model, and if such a threshold value is not present, then a generalized predetermined default value is employed [408]. Next the data is processed by removing [at 410] scan segments with Hounsfield data values outside expected values associated with the fiducial key values, following the collection [at 412] of the remaining points. If the data is empty [at 414], the CT value threshold is adjusted [at 416], the original value restored [at 418], and the segmenting processing scan segments continues [at 410]. Otherwise, with the existing data a center of mass is calculated [at 420], along with calculating [at 422] the X, Y, and Z axes. If the center of mass is not at the cross point of the XYZ axes [at 424], then the user is notified [at 426] and the process stopped [at 428]. If the center of mass is at the XYZ cross point then the data points are compared [430] with the designed fiducial data. If the cumulative error is larger than the maximum allowed error [at 432] then the user is notified [at 434] and the process ends [at 436]. If not, then the coordinate system is defined [at 438] at the XYZ cross point, and the scan profile is updated for the HU units [at 440].

Turning now to FIG. 4C, image information is obtained [442] from the tracker, being a suitable camera or other sensor. The image information is two-dimensional and is not required to be a stereo image pair. The image information may be sourced from a single imaging device in the tracker, or may be sourced from multiple imaging devices in the tracker. It bears pointing out that the presence of multiple imaging devices in a tracker does not automatically imply stereo imaging. The image information is analyzed [444] to determine whether a vectorized tracking marker is present in the image information. If not, then the user is queried [446] as to whether the process should continue or not. If not, then the process is ended [448]. If the process is to continue, then the user may be notified [450] that no tracking marker has been found in the image information, and the process returns to obtaining image information [442]. If a tracking marker has been found based on the image information, or one has been attached by the user upon the above notification [at 450], the offset and relative orientation of the tracking marker to the fiducial reference is obtained [452] from a suitable database. The term “database” is used in this specification to describe any source, amount or arrangement of such information, whether organized into a formal multi-element or multi-dimensional database or not. Such a database may be stored, for example, in system memory 217, fixed disk 244, or in external memory through network interface 248. A single data set comprising offset value and relative orientation may suffice in a simple implementation of this embodiment of the invention and may be provided, for example, by the user or may be within a memory unit of the controller or in a separate database or memory.

The offset and relative orientation of the tracking marker is used to define the origin of a coordinate system at the fiducial reference and to determine [454] the three-dimensional orientation of the fiducial reference based on the image information and the registration process ends [456]. In order to monitor the location and orientation of the fiducial reference in real time, the process may be looped back from step [454] to obtain new image information from the camera [at step 442]. A suitable query point may be included to allow the user to terminate the process. Detailed methods for determining orientations and locations of predetermined shapes or marked tracking markers from image data are known to practitioners of the art and will not be dwelt upon here. The coordinate system so derived is then used for tracking the motion of any items bearing vectorized tracking markers in the proximity of the surgical site. Other registration systems are also contemplated, for example using current other sensory data rather than the predetermined offset, or having a fiducial with a transmission capacity.

One example of an embodiment of the invention is shown in FIG. 5. In addition to passive vectorized fiducial key 502 mounted at a predetermined tooth and having a rigidly mounted passive vectorized tracking marker 504, an additional instrument or implement 506, for example a hand piece which may be a dental drill or scalpel, may be observed by a camera 508 serving as tracker of the monitoring system. Implement 506 may bear a vectorized tracking marker 507 allowing it to be tracked by tracker 508. Tracker 508 may in some embodiments be, in particular, a non-stereo tracker. Tracker 508 supplies image information of a field of view of tracker 508 to controller 520, which displays derived information on a display system or monitor 530. Controller 520 may be based on, for example, processor 214 and memory 217 of computer 210 of FIG. 2 and monitor 530 may have with controller 520 the structural relation that display screen 224 has with central processor 214 in FIG. 2.

In some embodiments, controller 520 may also control instrument or implement 506 and guide it to execute the surgical process based on image information that tracker 508 supplies to controller 520 and, thereby, on the scan data from an earlier scan. Such surgical processes are generally known as “robotic surgery”. As in the above embodiments, the image information of marker 504 allows determination of the three-dimensional location and orientation of fiducial marker 502 for which a prior scan has provided scan data for use by controller 520. In such embodiments, computer software stored in memory 217 of FIG. 2 is executed in for example processor 214 of computer 210 of FIG. 2 to guide instrument or implement 506 via, for example, I/O interface 218 of FIG. 2. Instrument or implement 506 may be linked to controller 520 via a wireless link or via a hardwired link (not shown in FIG. 5). Instrument or implement 506 may comprise a suitable actuator for moving a working point of instrument or implement 506 in order to execute the surgery. In such embodiments, instrument or implement 506 is referred to as a “robotic surgery instrument”.

Another example of an embodiment of the invention is shown in FIG. 6. Surgery site 600, for example a human stomach or chest, may have fiducial key 602 fixed to a predetermined position to support tracking marker 604. Other apparatus with suitable tracking markers may be in use in the process of the surgery at surgery site 600. By way of non-limiting example, endoscope 606 may have a further passive vectorized tracking marker 607, and biopsy needle 608 may also be present at surgery site 600 bearing a passive vectorized tracking marker 609. Sensor 610, serving as tracker for the system, may be for example a camera, infrared sensing device, or RADAR. In particular, the tracker may be a two-dimensional imaging tracker that produces a two-dimensional image of the surgery site 600 for use as image information for the purposes of embodiments of the invention, including two-dimensional image information of any vectorized tracking markers in the field of view of the tracker. Sensor 610 may be, for example, a non-stereo optical camera. In other embodiments sensor 610 may be a stereo camera. Surgery site 600, endoscope 606, biopsy needle 608, fiducial key 602 and vectorized tracking markers 604, 607 and 609 may all be in the field of view of tracker 610. Sensor 610 supplies image information of a field of view of sensor 610 to controller 520 which displays derived information on a display system or monitor 530.

In the embodiment of FIG. 6, controller 520 may also control biopsy needle 606 and guide it to execute the biopsy surgical process based on image information that tracker 610 supplies to controller 520 and, thereby, on the scan data from an earlier scan. The image information of marker 504 allows determination of the three-dimensional location and orientation of fiducial marker 502 for which a prior scan has provided scan data for use by controller 520. In such embodiments, computer software stored in memory 217 of FIG. 2 is executed in for example processor 214 of computer 210 of FIG. 2 to guide biopsy needle 606 via, for example, I/O interface 218 of FIG. 2. Biopsy needle 606 may be linked to controller 520 via a wireless link or via a hardwired link (not shown in FIG. 6). Biopsy needle 606 may comprise a suitable actuator for moving a working point of biopsy needle 606 in order to execute the biopsy surgery. In such embodiments, instrument or biopsy needle 606 is also referred to as a “robotic surgery instrument”.

In both of these robotic implementations the controller may operate on an autonomous basis, with human intervention being optional. Fiducial 502, 602 remains rigidly attached to the surgical site, and the marker 504, 604 remains in its fixed relative position and orientation with respect to fiducial 502, 602 if and when the patient moves. With both markers 504 and 507 in FIG. 5 tracked by tracker 508, or both markers 504 and 607 in FIG. 6 tracked by tracker 610, controller 520 may autonomously guide robotic instrument 506 or 606 respectively despite the motion of the patient. It bears repeating that, in cases where fiducial 502, 602 is directly visible to tracker 508, 610, fiducial 502, 602 may itself be vectorized with suitable markers bearing patterns that allow the spatial position and orientation of fiducial 502, 602 to be directly tracked by tracker 508, 610 without requiring separate tracking markers 504, 604 to be attached to fiducial 502, 602 using tracking poles.

The term “geometric information” is employed in the present specification to describe the collection of information regarding the shapes, sizes, perimeters, distribution, and the like of elements of the patterns on the tracking markers. The geometric information may include information on the pattern reference points of the patterns. A suitable pattern reference point on tracking marker 504 of FIG. 5 may be, for example without limitation, one of the four corners of the rectangle bearing the letter “C” on tracking marker 504. In implementations where the tracking markers bear pattern tags, the geometric information may include information regarding the patterns on the various pattern tags attached to the tracking markers and the associated locations of pattern reference points. The geometric information may also include the known spatial and orientation relationship between the tracking markers and pattern tags attached to the tracking markers.

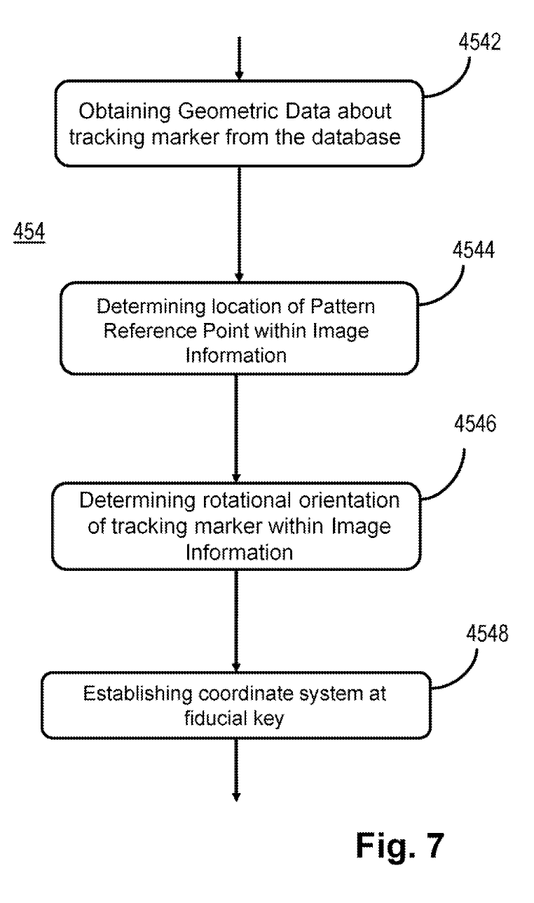

The automatic registration method for tracking surgical activity as per the present embodiment employing the pattern tags as described herein comprises the steps [402] to [456] of FIGS. 4A-C. In step [444] of FIG. 4C, tracking marker 12 has already been identified on the basis of its unique pattern. Step [454] of FIG. 4C will now be described in more detail at the hand of FIG. 7. The using [454] the offset and relative orientation of passive vectorized tracking marker 12 to define an origin of a coordinate system at fiducial key 10 and to determine the three-dimensional orientation of fiducial key 10 in image information, as shown in FIG. 4C, comprises the following steps in FIG. 7. The process starts with the controller, for example processor 214 and memory 217 of computer 210 of FIG. 2, obtaining [at 4542] from the database geometric information about at least one pattern tag associated with the tracking marker 12, the controller determining [at 4544] within the image information the location of at least one of the pattern reference points of the at least one pattern tag based on the geometric information, and the controller determining [at 4546] within the image information the rotational orientation of the at least one pattern tag based on the geometric information. With the relationship of the pattern reference point to tracking marker pre-established within the geometrical information, and the offset and relative orientation of the vectorized tracking marker 12 with respect to fiducial key 10 known (see step [452] in FIG. 4C), a coordinate system is established [at 4548] at the fiducial key 10.

The rotationally asymmetrical tracking marker arrangements described here may be applied to other fields of general machine vision and product tracking beyond the field of surgery. More specifically, while vectorized tracking marker 12 has been described in terms of being attached to fiducial key 10 by tracking pole 11 (see for example FIG. 3B), the patterned tracking markers of the present invention may be applied in other fields without the use of fiducials and tracking poles, in which case they are useful in determining the physical spatial orientation of items bearing the patterned tracking markers. By way of example, a flexible pattern tag may be applied to a cylindrical surface of an object, such as a can in the food industry. With the pattern reference point known and with the mathematical description of the pattern known, the position of the can and the curvature of the pattern tag may respectively be determined from image information obtained using a suitable tracker.

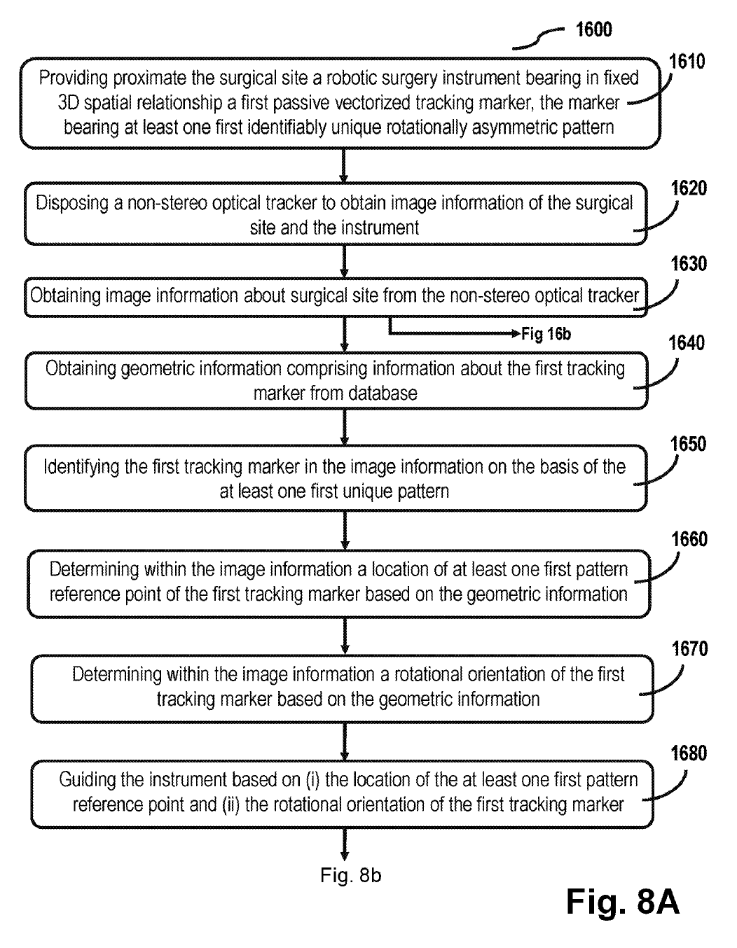

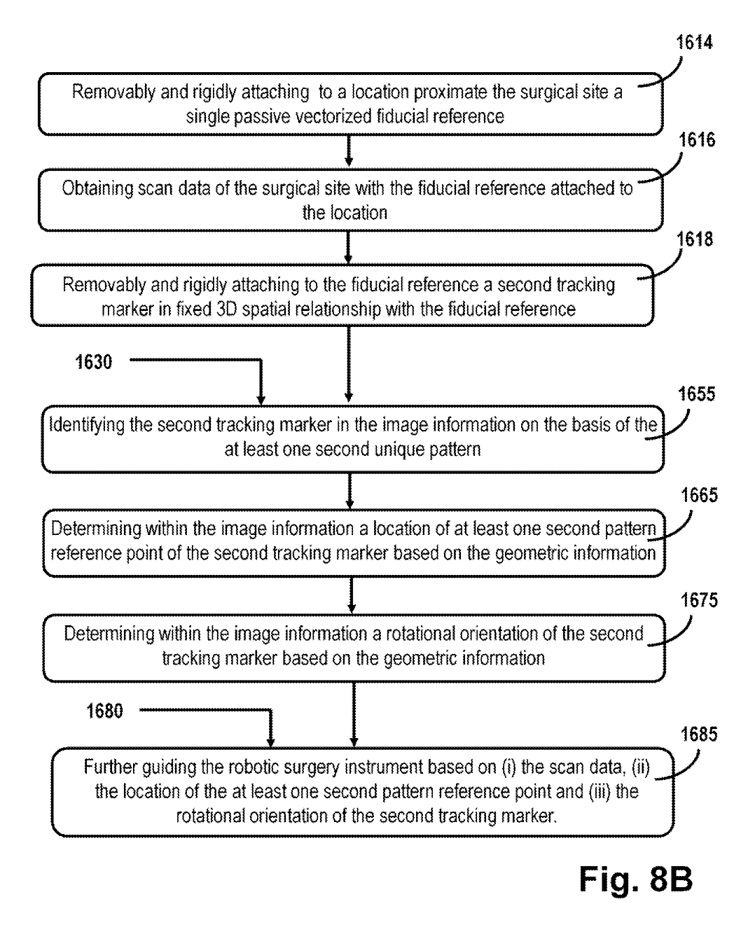

In a further aspect, as shown at the hand of the flow chart in FIGS. 8a and 8b, method [1600] is provided for guiding at a surgical site a robotic surgery instrument, the method comprising providing [1610] proximate the surgical site the robotic surgery instrument bearing in fixed three-dimensional spatial relationship with the instrument a first passive vectorized tracking marker, the marker bearing at least one first identifiably unique rotationally asymmetric pattern; disposing [1620] a non-stereo optical tracker to obtain image information of the surgical site and the instrument; obtaining image information [1630] about the surgical site from the non-stereo optical tracker; obtaining geometric information [1640] from a database, the geometric information comprising information about the first tracking marker; identifying [1650] the first tracking marker in the image information on the basis of the at least one first unique pattern; determining [1660] within the image information the location of at least one first pattern reference point of the first tracking marker based on the geometric information; determining [1670] within the image information the rotational orientation of the first tracking marker based on the geometric information; and guiding [1680] the robotic surgery instrument based on the location of the at least one first pattern reference point and the rotational orientation of the first tracking marker.

The geometric information may further comprise information about a second tracking marker bearing at least one second identifiably unique rotationally asymmetric pattern and the method may further comprise: removably and rigidly attaching [1614] to a location proximate the surgical site a single passive vectorized fiducial reference; obtaining [1616] scan data of the surgical area with the fiducial reference attached to the location, removably and rigidly attaching [1618] to the fiducial reference the second tracking marker in fixed three-dimensional spatial relationship with the fiducial reference, identifying [1655] the second tracking marker in the image information on the basis of the at least one second unique pattern; determining [1665] within the image information the location of at least one second pattern reference point of the second tracking marker based on the geometric information; determining [1675] within the image information a rotational orientation of the second tracking marker based on the geometric information; and further guiding [1685] the robotic surgery instrument based on the scan data, on the location of the at least one second pattern reference point, and on the rotational orientation of the second tracking marker.

In some implementations of the method, the fiducial reference may itself bear the second tracking marker, so that the step of attaching [1618] to the fiducial reference the second tracking marker in fixed three-dimensional spatial relationship with the fiducial reference is obviated.

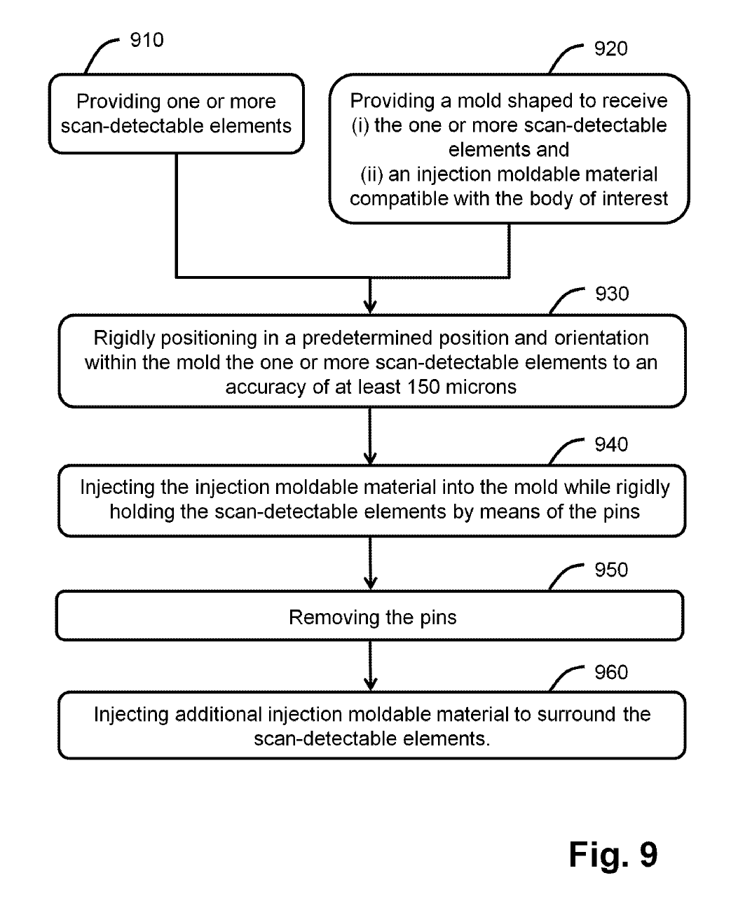

A method for manufacturing the multi-material fiducial references of FIGS. 3K, 3L and 3M may comprise, as shown in the flow chart in FIG. 9: providing [910] one or more scan-detectable elements of the above description; providing [920] a mold shaped to receive the one or more scan-detectable elements and an injection moldable material compatible with the body of interest; rigidly positioning [930] in a predetermined position and orientation within the mold the one or more scan-detectable elements by means of pins to an accuracy of at least 150 microns; injecting [940] the injection moldable material into the mold while rigidly holding the scan-detectable elements by means of the pins. The method may further comprise removing [950] the pins and further injecting [960] additional injection moldable material to surround the scan-detectable elements.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A user-calibration-free tracking system for monitoring the position and orientation of non-visible scan-detectable structure of a body of interest, the system comprising:
a vectorized fiducial reference adapted to be rigidly attached to the body of interest, the fiducial reference comprising a structural body composed of a structural material compatible with a material of the body of interest and one or more scan-detectable elements composed of a scan-detectable material rigidly embedded in the structural material wherein the one or more scan-detectable elements comprise a rotationally non-symmetric pattern;
a passive vectorized tracking marker rigidly attached to the fiducial reference at a predetermined location in a predetermined three-dimensional orientation with respect to the fiducial reference;
a non-stereo optical tracker arranged to obtain image information about an area encompassing at least a portion of the tracking marker;
a controller in communication with the tracker;
a display system in communication with the controller; and
previously obtained scan data of the body of interest with the fiducial reference fixed to the body showing the scan-detectable elements relative to the non-visible structure of the body of interest;
wherein the controller comprises a processor, a memory and a software program having a series of instructions which when executed by the processor determine the relative position and orientation of the marker and the one or more scan-detectable elements based on the image information and the scan data.

2. The tracking system of claim 1, further comprising a database, the database containing:
geometric information about the tracking marker; and
information about the rotationally non-symmetric pattern of the one or more scan-detectable elements.

3. The tracking system of claim 1, wherein the tracking marker is removably attached to the fiducial reference.

4. The tracking system of claim 3, wherein the tracking marker is attached to the fiducial reference via a tracking pole.

5. A fiducial reference for use in tracking a non-visible scan-detectable structure of a body of interest, the fiducial reference comprising:
a structural body composed of a structural material compatible with a material of the body of interest; and
one or more scan-detectable elements composed of a scan-detectable material rigidly embedded in the structural material;
wherein the one or more scan-detectable elements comprise a rotationally non-symmetric pattern.

6. The fiducial reference of claim 5, wherein the one or more scan-detectable elements are embedded in the structural material with an accuracy compatible with one of human and animal surgery.

7. The fiducial reference of claim 6, wherein the accuracy is a distance of 150 microns or less.

8. The fiducial reference of claim 6, wherein the accuracy is a distance of 80 microns or less.

9. The fiducial reference of claim 6, wherein the accuracy is a distance of 40 microns or less.

10. The fiducial reference of claim 6, wherein the accuracy is a distance of 16 microns or less.

11. The fiducial reference of claim 5, wherein the scan-detectable material has a radiographic density approximating a radiographic density of one of human and animal bone.

12. The fiducial reference of claim 5, wherein the scan-detectable material is one of a metal, a metallic-oxide ceramic, and silicon nitride.

13. The fiducial reference of claim 5, wherein the scan-detectable material is one of stainless steel, titanium, aluminum oxide, and zirconium oxide.

14. The fiducial reference of claim 5, further comprising a vectorized tracking marker.

15. The fiducial reference of claim 14, wherein the tracking marker bears an optically detectable rotationally asymmetric pattern.

16. The fiducial reference of claim 5, further comprising a locating hole for rigidly and removably attaching a vectorized tracking marker.

17. The fiducial reference of claim 16, further comprises a vectorized tracking marker associated with the locating hole wherein the tracking marker bears an optically detectable rotationally asymmetric pattern.

18. The fiducial reference of claim 16, wherein the tracking marker is attachable to the fiducial by means of a tracking pole.

19. A method for manufacturing a multi-material fiducial reference for tracking a non-visible scan-detectable structure of a body of interest, the method comprising:
providing one or more scan-detectable elements;
providing a mold shaped to receive the one or more scan-detectable elements and an injection moldable material compatible with the body of interest;
rigidly positioning in a predetermined position and orientation within the mold the one or more scan-detectable elements by at least one pin to an accuracy of at least 150 microns; and
injecting the injection moldable material into the mold while rigidly holding the scan-detectable elements by the at least one pin.

20. The method of claim 19, further comprising:
removing the at least one pin; and
further injecting additional injection moldable material to surround the scan-detectable elements.
